(19) United States
(12) Patent Application Publication
(10) Pub. No.: US 2025/0360282 A1
(43) Pub. Date: Nov. 27, 2025

(54) GAS REMIX STIMULATOR

(71) Applicant: Emercent Technologies LLC, Rochester, MN (US)

(72) Inventors: J. Hunter Downs, III, Rochester, MN (US); Bruce D. Johnson, Rochester, MN (US)

(73) Assignee: Emercent Technologies LLC, Rochester, MN (US)

(21) Appl. No.: 19/216,635

(22) Filed: May 22, 2025

Related U.S. Application Data
(60) Provisional application No. 63/650,433, filed on May 22, 2024.

(51) Int. Cl. A61M 16/12; A61M 16/00; A61M 16/08

(52) U.S. Cl. CPC A61M 16/12; A61M 16/0003; A61M 16/0875; A61M 16/0883; A61M 2202/0007; A61M 2202/0225; A61M 2205/3327; A61M 2205/3331; A61M 2210/0625; A61M 2230/43

(57) ABSTRACT

A system may include an air manifold, a reservoir, a coupler and a straw. The air manifold may have three terminal ends—an atmosphere end, a respiratory end and a mixing end. The air manifold may further include four ports—an atmosphere port disposed at the atmosphere end, an atmosphere-mixing port disposed at the mixing end, a respiratory port disposed at the respiratory end, and a respiratory-mixing port disposed at the mixing end. The atmosphere port may be fluidly coupled to the atmosphere mixing port, the respiratory port may be fluidly coupled to the respiratory-mixing port, and the respiratory port and atmosphere port may be fluidly isolated from each other within the air manifold. The coupler may be configured to couple the air manifold to the reservoir. The straw may removably coupled to the respiratory port and extend into the reservoir.

### CROSS REFERENCE TO RELATED APPLICATIONS

This application claims priority to U.S. Application Ser. No. 63/650,433, titled “Gas Remix Stimulator,” filed on May 22, 2024. This application incorporates the entire contents of the foregoing application herein by reference.

### TECHNICAL FIELD

Various implementations relate generally to devices and methods for temporarily inducing hypercapnia for diagnostic or other purposes.

### BACKGROUND

The following brief overview of a number of distinct medical concepts may be useful context for the description that follows.

Hypercapnia is a state of excessive carbon dioxide (CO₂) in the bloodstream. In many cases, hypercapnia is caused by inadequate respiration or inadequate transfer, during respiration, of CO₂ from the bloodstream to exhaled air in the lungs.

Hypoxia is a state of insufficient oxygen at the tissue level to sustain homeostasis (i.e., dynamic equilibrium of critical cell processes, including, for example, communication between cells, energy production and metabolism, waste removal, temperature regulation, regulation of water balance, pH balance, ion and electrolyte balance, etc.).

Living systems generally thrive in states of normocapnia (normal, healthy concentrations of CO₂, rather than hypercapnia or hypocapnia (low levels of CO₂)) and normoxia (normal, healthy concentrations of oxygen, rather than hypoxia or hyperoxia (too much oxygen)). However, certain beneficial processes may be stimulated by short periods of hypercapnia or hypoxia.

Cerebrovascular reactivity (CVR) is the ability of blood vessels in the brain to dilate or constrict to adjust blood flow in response to changing conditions, to maintain blood flow across the brain. For example, dilation of cerebral vessels can be important to increase blood flow during periods of hypercapnia; and CVR may be tested by inducing brief periods of hypercapnia in a subject. Such tests can be helpful in assessing the subject's ability to engage in exercise, when CO₂ levels are naturally elevated.

Efficient CVR ensures that increased demands for oxygen and nutrients during exercise are met—facilitating high cognitive functioning and high overall physical performance. In contrast, impaired CVR may reduce exercise performance, decrease endurance and quicken the onset of fatigue. More significantly, impaired CVR may be associated with hypertension and diabetes. For many, CVR may be maintained or improved through regular aerobic exercise, managing cardiovascular risk factors and adopting an overall healthy lifestyle.

Transcription factors are proteins that help regulate gene expression by binding to or near specific genes to control various biological processes, including cell growth and development and response to environmental stimuli. For example, transcription factors may activate stress response genes when a subject is facing adverse conditions (e.g., to support a “fight or flight” response) or metabolic genes in response to changes in nutritional intake of a subject.

Hypoxia-inducible factor (HIF) is a particular group of transcription factors that activates certain genes under low oxygen conditions, which can enable cells to adapt to (and survive through) periods of hypoxia. HIFs are key regulators in the formation of new blood vessels (angiogenesis), the production of red blood cells (erythropoiesis) and metabolism (enabling maintenance of oxygen homeostasis).

The endothelium is the thin layer of cells lining the interior surface of blood vessels, and this layer of cells plays a critical role in overall cardiovascular health and function. Specifically, the endothelium plays a significant role in the dilation and constriction of blood vessels to regulate blood flow and pressure—for example, by releasing either vasodilators or vasoconstrictors. The endothelium also functions as a selective barrier between the bloodstream and surrounding tissue to control homeostasis, immune response and inflammation—for example, by expressing and releasing various cytokines and adhesion molecules to recruit white blood cells to areas that may require an immune response. The endothelium also plays a role in either inhibiting blood clotting—for example, by producing and selectively secreting clotting inhibitors such as a thrombomodulin or heparin sulfate, or by promoting blood clotting by producing and selectively secreting von Willebrand factor or plasminogen activator inhibitor. The endothelium also responds to mechanical stimuli (e.g., shear stress associated with blood flow) and may influence vascular remodeling and gene expression within the endothelial cells. Impaired endothelial function can indicate a higher risk for vascular diseases such as hypertension, diabetes, hypercholesterolemia (an excess of cholesterol in the bloodstream), heart attack and stroke.

In some instances, hypercapnia may be used to indirectly assess endothelial function-especially through its impact on CVR. That is, induced hypercapnia may be used to trigger the endothelium's natural vasodilatory response to increased CO₂, which response is partly mediated by nitric oxide, a key endothelial-derived relaxing factor. CVR can be measured through techniques such as transcranial Doppler ultrasound or functional MRI (fMRI)—both techniques of which may be employed to assess changes in blood flow or blood volume in the brain in response to the induced hypercapnia. A quick response in blood flow or blood volume in the brain following induced hypercapnia can imply a healthy endothelium; whereas a delayed or absent response following induced hypercapnia can imply endothelial dysfunction-which can be a risk factor for cardiovascular and cerebrovascular diseases.

### SUMMARY

In some implementations, a system includes an air manifold, a reservoir, a coupler and a straw. The air manifold may have three terminal ends, including an atmosphere end, a respiratory end and a mixing end. The air manifold may further include four ports, including an atmosphere port disposed at the atmosphere end, an atmosphere-mixing port disposed at the mixing end, a respiratory port disposed at the respiratory end, and a respiratory-mixing port disposed at the mixing end. The atmosphere port may be fluidly coupled to the atmosphere mixing port, the respiratory port may be fluidly coupled to the respiratory-mixing port, and the respiratory port and atmosphere port may be fluidly isolated from each other within the air manifold. The coupler may have a first end and a second end and an open interior between the first end and the second end. The first end may be configured to couple to the mixing end, and the second end may be configured to couple to the reservoir. The straw may be configured to be removably coupled to the respiratory port and to extend through the coupler and into the reservoir.

The open interior may have a progressively decreasing diameter from the second end toward the first end, such that a first reservoir may be removably coupled by the coupler close to the second end, and a second reservoir with a smaller-diameter neck than the first reservoir may be removably coupled to the coupler closer to the first end.

The system may further include a mouthpiece having a mouth interface on one end, a respiratory interface on another end and a channel therebetween that fluidly couples the mouth interface to the respiratory interface. The respiratory interface may be configured to be removably coupled to the respiratory end, and the mouth interface may be configured to be secured by a user's mouth. In some implementations, the mouthpiece includes one or more bite plates.

The system may further include a restrictor plate that is configured to be coupled to the atmosphere end, wherein the restrictor plate comprises an opening that has less cross-sectional area than a cross-sectional area of the atmosphere port. A system or kit may include a sanitizing spray or sanitizing fluid to facilitate sanitization of the one or more restrictor plates or the plurality of reservoirs.

In some implementations, a kit may include multiple systems, each system with an air manifold, a reservoir, a coupler and a straw. The kit may include a plurality of reservoirs, where each reservoir in the plurality of reservoirs has a unique volume capacity. The kit may include a restrictor plate that is configured to be coupled to the atmosphere end and that includes an opening that has less cross-sectional area than a cross-sectional area of the atmosphere port. A plurality of restrictor plates may be provided, each of which may be configured to be coupled to the atmosphere end, wherein each restrictor plate in the plurality of restrictor plates has a different reduction in cross-sectional area relative to a cross-sectional area of the atmosphere port.

A kit may include a CO₂ sensor to facilitate selection of a reservoir from among the plurality of reservoirs for a particular patient. The CO₂ sensor may be configured to be at least partially disposed in an interior of the one reservoir. Each reservoir in the plurality of reservoirs may include a CO₂ sensor that is integrated into the reservoir or removably attachable to the reservoir to measure CO₂ in an interior of the reservoir.

### BRIEF DESCRIPTION OF THE DRAWINGS

FIG. 1 illustrates various components an exemplary system.

FIG. 2 illustrates the components shown in FIG. 1 assembled in an exemplary configuration.

FIG. 3 illustrates an optional mouthpiece.

FIG. 4 illustrates an optional flow restrictor.

FIG. 5 provides additional detail of an exemplary air manifold.

FIGS. 6A-6D depict two inhalation and exhalation cycles using an exemplary system.

### DETAILED DESCRIPTION

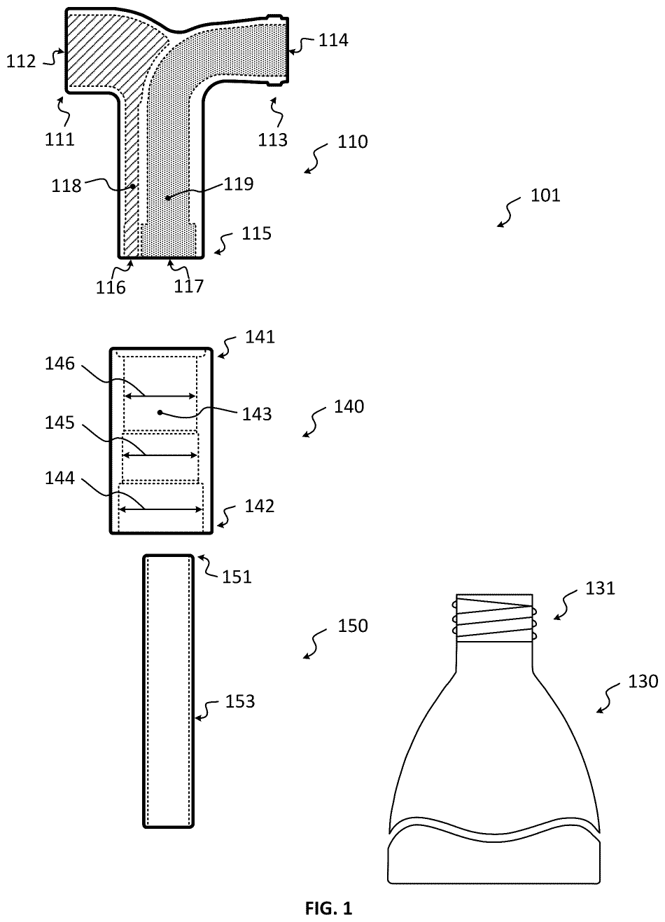

FIG. 1 illustrates an exemplary system 101 that includes an air manifold 110, a reservoir 130, a coupler 140 and a straw 150. In the implementation shown, the air manifold 110 includes three terminal ends: an atmosphere end 111, a respiratory end 113, and a mixing end 115; and the air manifold 110 further includes four ports: an atmosphere port 112, disposed at the atmosphere end 111; an atmosphere-mixing port 116, disposed at the mixing end 115; a respiratory port 114, disposed at the respiratory end 113; and a respiratory-mixing port 117, also disposed at the mixing end 115. As shown, the atmosphere port 112 and the atmosphere-mixing port 116 are fluidly coupled within the air manifold 110 by an atmosphere channel 118; the respiratory port 114 and respiratory-mixing port 117 are fluidly coupled within the air manifold 110 by a respiratory channel 119; but the atmosphere port 112 and the respiratory port 114 are fluidly isolated from each other within the air manifold 110.

The reservoir 130 may be any vessel capable of storing a gas. In some implementations, as shown, the reservoir 130 may have the form of a bottle, with a neck 131; and walls of the bottle may be substantially inelastic. In other implementations, the reservoir 130 may have walls that are elastic and resilient, being capable of being inflated under pressure. The reservoir 130 may have any suitable volume (e.g., 6 liters, 4 liters, 2 liters, 1 liter, 0.5 liters, etc.). In some implementations, a kit may be provided that includes multiple reservoirs, each with a different volume (e.g., a kit may include a 6-liter reservoir, a 4-liter reservoir, a 2-liter reservoir and a 1-liter reservoir).

The coupler 140 has a first end 141 and a second end 142 and an open interior 143 between the first end 141 and the second end 142. The first end 141 may be configured to be coupled to the mixing end 115 of the air manifold 110; and the second end 142 may be configured to be coupled to the reservoir 130 (e.g., specifically, the neck 131 of the reservoir 130). In some implementations, as shown, the open interior 143 may have dimensions that are staged—for example, with a progressively smaller diameters (e.g., a first diameter 144, a second diameter 145 that is smaller than the first diameter 144, and a third diameter 146 that is smaller than the second diameter 145)—to facilitate coupling with reservoirs having necks of various sizes. Some implementations may have more or fewer separate staged sections, and some implementations may have a smooth, continuously decreasing diameter.

The straw 150 may be configured to be removably coupled to the respiratory port 117 on one end 151, with the straw body 153 extending, during use, through the coupler 140 and into the reservoir 130.

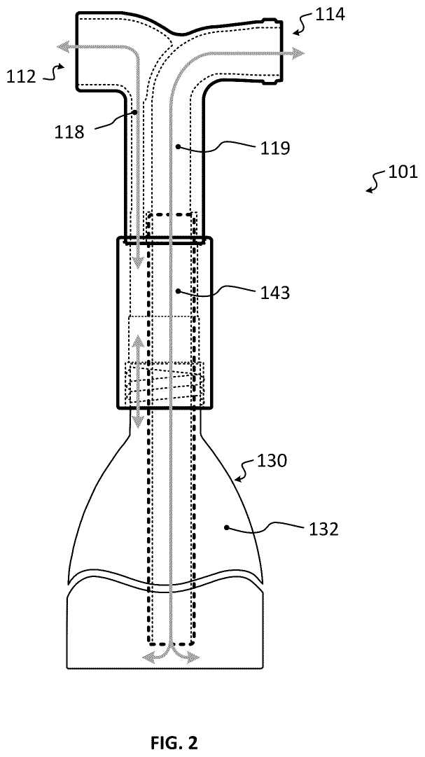

FIG. 2 illustrates the components shown in FIG. 1 assembled in an exemplary manner. In some implementations, components that may be removably coupled are configured to seal against each other, such that channels or cavities within adjacent, coupled components are fluidly coupled to each other and fluidly isolated from areas exterior to the adjacent, coupled components. For example, the respiratory channel 119 may be fluidly coupled to the open interior 143 and to an interior 132 of the reservoir, but fluidly isolated from an exterior of the system 101, except through the respirator port 114 or the atmosphere port 112. As depicted by the arrows, atmospheric air may flow through the atmosphere port 112, through the atmosphere channel 118 and the open interior 143 and into and out of the interior 132 of the reservoir 130; respiratory air (inhaled air and exhaled air of a user) may flow through the respiratory channel 119 and open interior 143 and into and out of the interior 132 of the reservoir 130.

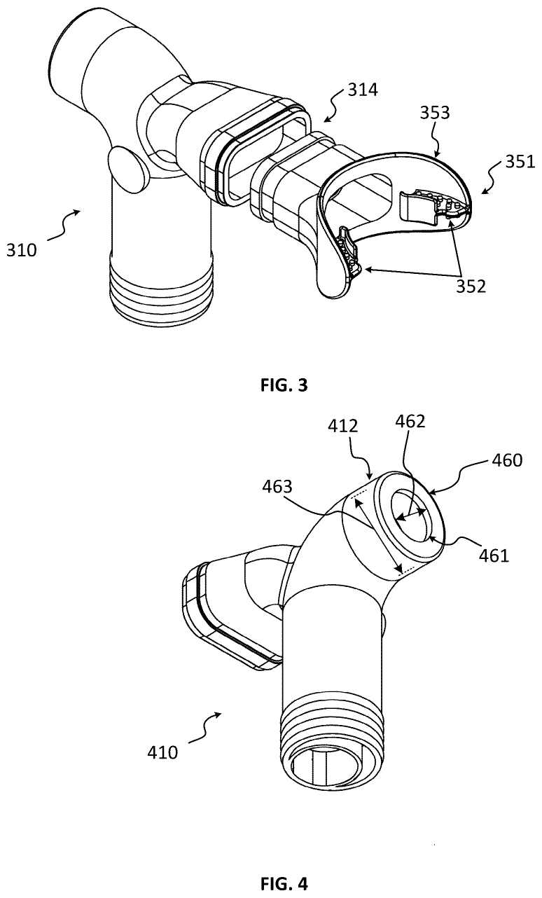

FIG. 3 illustrates additional details of an exemplary air manifold 310. In some implementations, as shown, a mouthpiece 351 may be provided that may removably couplable to a respiratory port 314 on the air manifold 310. The mouthpiece 351 may be configured to be placed partially in a user's mouth, with bite plates 352 that the user can grip with his or her teeth, and a shield 353 configured to be flush against the user's lips and cheeks. In such implementations, the user may inhale and exhale through the mouthpiece 351.

FIG. 4 illustrates a restrictor plate 460 that may be provided with an exemplary air manifold 410. In some implementations, the restrictor plate 460 has an opening 461 with a smaller diameter 462 than the diameter 463 associated with the atmosphere port 412. In such implementations, the restrictor plate 460 may impede, or add resistance to, flow of air from an area external to and surrounding the air manifold 410. In some implementations, a kit may be provided that includes a plurality of flow restrictors (e.g., flow restrictors that restrict flow therethrough by 5%, 10%, 20%, 30%, etc.).

By controlling various parameters of the system, including, for example, an appropriate restrictor plate 460, reservoir size and straw length, and time of use, a user may selectively control a mix of atmospheric air and exhaled air, thereby controlling a level of CO₂ in the reservoir. In some implementations, a restrictor plate 460 may be removable, and other restrictor plates with different diameter openings may be provided, such that a user can configure flow into and out of the air manifold 410.

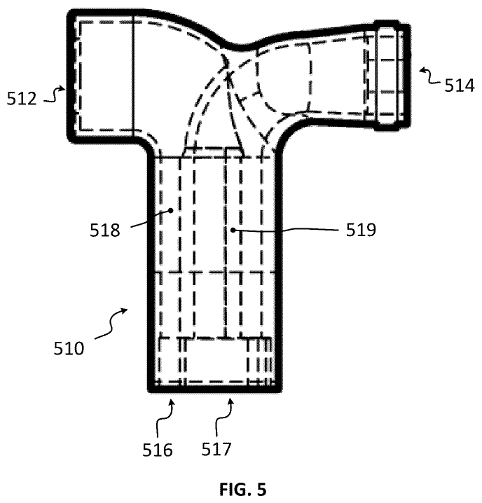

FIG. 5 illustrates additional cross-sectional detail of an exemplary air manifold 510—in particular, illustrating the internal contours of an atmosphere channel 518 coupling an atmosphere port 512 and an atmosphere-mixing port 516, and a respiratory channel 519 coupling a respiratory port 514 and a respiratory-mixing port 517.

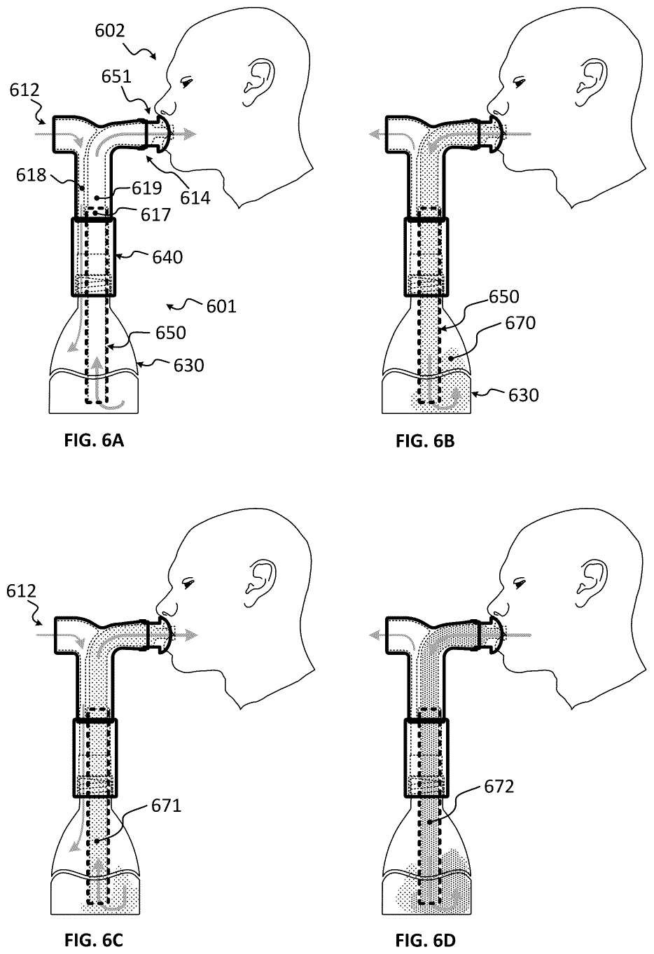

FIGS. 6A-6D illustrate an exemplary system 601 in use. In particular, FIG. 6A depicts a user 602 using a system with a mouthpiece 651. The user 602 has his mouth pressed against the mouthpiece 651, and may be biting on bite plates (not shown) to maintain sealed contact with the mouthpiece 651. As the user 602 inhales, air is drawn into the straw 650 from within a reservoir 630. The inspiratory action of the user 602 draws air through the straw 650, through the coupler 640, through a respiratory-mixing port 617, through a respiratory channel 619, through the respiratory port 614, through the mouthpiece 651 and into the lungs of the user 602. Air that is initially inspired primary comes from the reservoir 630, but as depicted, some atmospheric air is sucked into the reservoir 630 through the atmosphere port 612, atmosphere channel 618, coupler 640 and into the top of reservoir 630.

FIG. 6B depicts the user 602 exhaling. The exhaled air 670 is depicted with shading, representing its higher concentration CO₂ (e.g., about 4%, rather than 0.04% in atmospheric air). Notably, in some implementations, as shown, atmospheric air enters the reservoir 630 at the top, whereas air is drawn into and exhaled from the straw 650 at the bottom of the reservoir 630. Such implementations may promote mixing within the reservoir 630.

FIG. 6C depicts the user 602 again inhaling, after exhaling. As depicted, the subsequently inhaled air 671 has a higher concentration of CO₂ from the last exhalation(s), though some fresh air may be drawn into the atmosphere port 612 with each inhalation, and some air may be exhausted from the reservoir 630 through the atmosphere port 612 with each exhalation.

FIG. 6D depicts the user 602 again exhaling. As depicted, subsequently exhaled air 672 may have an even higher concentration of CO₂ than previously exhaled air 670, and the exhaled air and atmosphere air may continue mixing in the reservoir 630.

In some implementations, an equilibrium mixture of CO₂ may be reached, which may depend on the volume of the reservoir 630, the length of the straw 650, the size of the atmosphere channel 618, the size of any restrictor (e.g., a restrictor 460, like that shown in FIG. 4) that may be present at the respiratory port 612, etc.; in other implementations, CO₂ may continue increasing throughout use, like that depicted in FIGS. 6A-6D. In such implementations, it may be necessary to limit time of use to avoid inducing dangerous levels of hypercapnia.

Several implementations have been described with reference to exemplary aspects, but it will be understood by those skilled in the art that various changes may be made and equivalents may be substituted for elements thereof without departing from the contemplated scope. For example, various mouthpieces, masks and filters may be attached to the respiratory port; an adjustable aperture may be attached at the atmosphere port; sensors may be added at various points (e.g., at or near the respiratory port) to measure the flow and gas content entering the lungs of a user; a kit may be provided that includes a sensor in the reservoir that can be used by a clinician to calibrate an ideal equilibrium mix for a specific patient be facilitating a selection of an appropriate reservoir volume and/or an appropriate flow restrictor; in some implementations, a kit may include a plurality of systems (e.g., for a number of discrete patients) with a calibration sensor that can be employed by a clinician to a calibrate a plurality of individual patients; a sensor (e.g., a CO₂ sensor) may be provided in all reservoirs (e.g., integrated into the reservoir, or removably attachable to the reservoir to measure CO₂ in the interior of the reservoir); a kit with multiple systems may include a sanitizing spray or fluid to facilitate cleaning by clinician staff of reservoirs, flow restrictors or other components; apertures or other connections may be added to the reservoir to facilitate the introduction of other gases or inhalants that may have lung function measurement or therapeutic benefits; desiccants or adsorbents may be employed to capture particulates or specific gases; an aperture may be added between the atmosphere port and the respiratory port to bypass some of the mixing that would otherwise take place within the reservoir. In general, “about,” “approximately” or “substantially” may mean within 1%, or 5%, or 10%, or 20%, or 50%, or 100% of a nominal value.

Many other variations are possible, and modifications may be made to adapt a particular situation or material to the teachings provided herein without departing from the essential scope thereof. Therefore, it is intended that the scope include all aspects falling within the scope of the appended claims.

What is claimed is:

1. A system comprising:
an air manifold with three terminal ends, including an atmosphere end, a respiratory end and a mixing end; the air manifold further including four ports, including an atmosphere port disposed at the atmosphere end, an atmosphere-mixing port disposed at the mixing end, a respiratory port disposed at the respiratory end, and a respiratory-mixing port disposed at the mixing end; wherein the atmosphere port is fluidly coupled to the atmosphere mixing port, the respiratory port is fluidly coupled to the respiratory-mixing port, and the respiratory port and atmosphere port are fluidly isolated from each other within the air manifold;
a reservoir;
a coupler having a first end and a second end and an open interior between the first end and the second end, the first end being configured to couple to the mixing end, and the second end being configured to couple to the reservoir; and
a straw that is configured to be removably coupled to the respiratory port and to extend through the coupler and into the reservoir.

2. The system of claim 1, wherein the open interior has a progressively decreasing diameter from the second end toward the first end, such that a first reservoir may be removably coupled by the coupler close to the second end, and a second reservoir with a smaller-diameter neck than the first reservoir may be removably coupled to the coupler closer to the first end.

3. The system of claim 1, further comprising a mouthpiece having a mouth interface on one end, a respiratory interface on another end and a channel therebetween that fluidly couples the mouth interface to the respiratory interface; wherein the respiratory interface is configured to be removably coupled to the respiratory end, and wherein the mouth interface is configured to be secured by a user's mouth.

4. The system of claim 3, wherein the mouthpiece comprises one or more bite plates.

5. The system of claim 1, further comprising a restrictor plate that is configured to be coupled to the atmosphere end, wherein the restrictor plate comprises an opening that has less cross-sectional area than a cross-sectional area of the atmosphere port.

6. A kit comprising:
an air manifold with three terminal ends, including an atmosphere end, a respiratory end and a mixing end; the air manifold further including four ports, including an atmosphere port disposed at the atmosphere end, an atmosphere-mixing port disposed at the mixing end, a respiratory port disposed at the respiratory end, and a respiratory-mixing port disposed at the mixing end; wherein the atmosphere port is fluidly coupled to the atmosphere mixing port, the respiratory port is fluidly coupled to the respiratory-mixing port, and the respiratory port and atmosphere port are fluidly isolated from each other within the air manifold;
a plurality of reservoirs, each reservoir in the plurality having a unique volume capacity;
a coupler having a first end and a second end and an open interior between the first end and the second end, the first end being configured to couple to the mixing end, and the second end being configured to couple to one reservoir in the plurality of reservoirs; and
a straw that is configured to be removably coupled to the respiratory port and to extend through the coupler and into the one reservoir.

7. The kit of claim 6, further comprising a restrictor plate that is configured to be coupled to the atmosphere end, wherein the restrictor plate comprises an opening that has less cross-sectional area than a cross-sectional area of the atmosphere port.

8. The kit of claim 6, further comprising a plurality of restrictor plates, each of which is configured to be coupled to the atmosphere end, wherein each restrictor plate in the plurality of restrictor plates has a different reduction in cross-sectional area relative to a cross-sectional area of the atmosphere port.

9. The kit of claim 6, further comprising a CO₂ sensor to facilitate selection of a reservoir from among the plurality of reservoirs for a particular patient.

10. A kit comprising:
an air manifold with three terminal ends, including an atmosphere end, a respiratory end and a mixing end; the air manifold further including four ports, including an atmosphere port disposed at the atmosphere end, an atmosphere-mixing port disposed at the mixing end, a respiratory port disposed at the respiratory end, and a respiratory-mixing port disposed at the mixing end; wherein the atmosphere port is fluidly coupled to the atmosphere mixing port, the respiratory port is fluidly coupled to the respiratory-mixing port, and the respiratory port and atmosphere port are fluidly isolated from each other within the air manifold;
a plurality of reservoirs, each reservoir in the plurality having a unique volume capacity;
a coupler having a first end and a second end and an open interior between the first end and the second end, the first end being configured to couple to the mixing end, and the second end being configured to couple to one reservoir in the plurality of reservoirs;
a straw that is configured to be removably coupled to the respiratory port and to extend through the coupler and into the one reservoir; and
one or more restrictor plates, each of the one or more restrictor plates having a opening therethrough that reduces the cross-sectional area relative to a cross-sectional area of the atmosphere port.

11. The kit of claim 10, further comprising a CO₂ sensor to facilitate selection of a reservoir from among the plurality of reservoirs for a particular patient.

12. The kit of claim 11, wherein the CO₂ sensor is configured to be at least partially disposed in an interior of the one reservoir.

13. The kit of claim 10, wherein each reservoir in the plurality of reservoirs includes a CO₂ sensor that is integrated into the reservoir or removably attachable to the reservoir to measure CO₂ in an interior of the reservoir.

14. The kit of claim 10, further comprising a sanitizing spray or sanitizing fluid to facilitate sanitization of the one or more restrictor plates or the plurality of reservoirs.
